(19) United States
(12) Patent Application Publication
(10) Pub. No.: US 2023/0173122 A1
(43) Pub. Date: Jun. 8, 2023

(54) REUSABLE CONTAINER FOR BULK PROCESSING IN HIGH PRESSURE APPLICATIONS

(71) Applicant: Avure Technologies Incorporated, Middletown, OH (US)

(72) Inventors: Rick D. Pelfrey, Bellbrook, OH (US); Ryan Pereira, Middletown, OH (US); Limin Zhang, Mason, OH (US)

(73) Assignee: Avure Technologies Incorporated, Middletown, OH (US)

(21) Appl. No.: 17/906,461

(22) PCT Filed: Mar. 24, 2021
(86) PCT No.: PCT/US2021/023993

§ 371 Date: Sep. 15, 2022

Related U.S. Application Data
(60) Provisional application No. 63/001,119, filed on Mar. 27, 2020.

(51) Int. Cl. A61L 2/26; A61L 2/02; A61L 2/00; B65D 88/06; B08B 9/08; A23L 3/00; A23L 3/015

(52) U.S. Cl. CPC A61L 2/26; A61L 2/02; A61L 2/0011; B65D 88/06; B08B 9/0804; A23L 3/001; A23L 3/0155; A61L 2202/182; B08B 2209/08

(57) ABSTRACT

A bulk HPP container (10) includes a flexible body portion (12) having closed off ends (14), at least one of which is depicted as being recessed to enable the containers (10) to be positioned end-to-end in an efficient manner, for example, when placed into an HPP pressure vessel, such as a wire wound vessel. One or more openings are provided in the body portion of the container with n appropriate closure for pumpable product to enter and exit the container. In this regard, an inlet valve (16) or other type of closure may be located at the opening in one or both of the ends of the container. Also, one or more outlet valves (18) or other type of closure are located at an opening on the body portion (12) of the container for emptying the container, for example, after HPP. The body portion 12) is composed of sufficient flexural strength and sufficient flexural modulus to enable the container to reduce in volume by from 0 to at least 30% while being rigid enough for reuse over a desired number of HPP cycles.

### CROSS-REFERENCE TO RELATED APPLICATION

This patent application claims priority of U.S. Patent Application Ser. No. 63/001,119, filed on Mar. 27, 2020, the entire disclosure of which is hereby incorporated by reference herein for all purposes.

### BACKGROUND

High pressure processing (HPP) is used to reduce the microbial load on foods, beverages, cosmetics, pharmaceuticals, and other products without altering the characteristics of the processed product. The pressure level required for HPP to be successful is typically at least 4,000 bar.

Traditional equipment for treatment of beverages and other liquids as well as pumpable foods and other substances by HPP is based on the processing of the pumpable products after having been placed as individual units into flexible containers or packaging, for example, bottles, cartons or pouches. In the traditional form of HPP, such individual units are grouped or consolidated within a larger reusable load basket which is sized and shaped to fit into a high pressure vessel. An example of such a load basket is disclosed by PCT/US2017/065842 (International Patent Application Publication No. WO 2018/111891).

Such high pressure vessel is filled with water which serves as the pressurizing medium. Once the pressure vessel has been filled and closed, high capacity pumps introduce additional water into the pressure vessel so that the pressure therein ranges from about 4,000 to 10,000 bar. This pressure is maintained for a sufficient length of time, from a few seconds to several minutes, to reduce the microbial load on the products being treated. The particular pressure level and the time duration of such pressure are specific to the product being processed.

Once the desired level of inactivation of the microorganisms has been achieved, the pressure in the vessel is released and the load basket is removed from therein so that the individual packages or units can be extracted. The processed product has, after being exposed to high pressure and hold time, been pasteurized, the microbial load has been reduced, and an extended shelf life has been achieved.

One drawback of using the HPP process with individual packages or units is that a relatively low proportion of the useful volume of the pressure vessel is employed, typically from only 50-80% depending on, for example, the size and geometry of the pressure vessel as well as the size and shape of the individual product units. Also, by processing the treated product in its final packaging there is a limitation on the type and design of such packaging. For example, such packaging must be flexible to isostatically compress under HPP and also be able to withstand the high water pressures of HPP. As such, packaging composed of glass or other incompressible material is not suitable for HPP.

Further, each individual package or unit must be handled when loading in the load basket and unloaded therefrom prior and after HPP. As such, there is a need to develop HPP systems that use a larger proportion of the available volume of the high pressure vessel, eliminate the need to handle individual packages or units during HPP, and also enable a wider variety of final product packaging to be employed.

Currently, one method being used to seek to address the foregoing limitation of using HPP with products in their final packaging is to instead bulk process the product, especially pumpable products. The pumpable products are placed in a large flexible bag or bladder type container located inside the pressure vessel thereby to occupy a larger proportion of the useful volume within the pressure vessel. However, the usefulness and success of such bladders or flexible bags is limited by operational deficiencies due to service and maintenance challenges associated with bladders or flexible bags.

Thus, there is a need for an HPP bulk container that is compatible with pumpable products and that is easily serviceable. Other requirements are to be able to fill and empty the bulk container outside of the pressure vessel, as well as the container being durable and rigid enough for repeated usage of many cycles. In this regard, the bulk container must be sufficiently rigid to withstand material handling operations, but also flexible enough to enable pressure transfer to the product during HPP.

Moreover, the inlet and outlet valves or other types of closures for the bulk container must be integrated to the container and be robust enough to securely seal the container during HPP while also being durable enough to not require frequent repair or replacement. The closures must also ensure rapid and uninterrupted filling and emptying of the bulk container. The present disclosure seeks to provide an HPP bulk container that meets the foregoing criteria.

### SUMMARY OF THE INVENTION

In accordance with one embodiment of the present disclosure a reusable container for holding pumpable materials during high pressure processing of the material, the container includes:

a body for receiving the pumpable material and for holding the material during high pressure processing, the body constructed to retain its shape whether empty and filled and having sufficient flexibility to decrease in volume to match the reduction in volume of the held material during high pressure processing;

at least one opening in the body through which the pumpable material is received into the container and/or removed from the container;

a closure at the at least one opening;

the body is configured to be of sufficient strength combined with flexibility to function as both reusable container and self-supporting carrier during all stages of high pressure processing and to maintain its diameter, length, and volume properties for an indefinite number of cycles that is more than achieved during one day of operation.

In any of the embodiments described herein, the body is of sufficient flexibility to reduce in volume by 0 to at least 30 percent during high pressure processing.

In any of the embodiments described herein, wherein the closure located at at least one of the openings, when in closed position is within the outer envelope of the container body.

In any of the embodiments described herein, wherein the closure located at at least one of the openings does not extend beyond the overall length or width of the container body

In any of the embodiments described herein, wherein the body in cross section is selected from cylindrical, pentagonal, hexagonal, and octagonal.

In any of the embodiments described herein, wherein the body defines a length with end portions, and with one or both of the end portions recessed.

In any of the embodiments described herein, wherein one or both end(s) of the body are convex in the direction into the body.

In any of the embodiments described herein, wherein one or both end(s) of the body are in the shape of a portion of a sphere.

In any of the embodiments described herein, wherein an opening is located at at least one end portion of the body.

In any of the embodiments described herein, wherein the body is composed of a material selected from the group consisting of a metal and a polymer.

In any of the embodiments described herein, wherein the polymer material from which the body is composed is of a material thickness sufficient to enable the container to retain its shape when filled with product to be processed by HPP and to reduce in volume by 0 to at least 30 percent, or what is needed for the product being processed, and to withstand a desired number of HPP cycles.

In any of the embodiments described herein, wherein the body has a flexural strength sufficient to enable the container to retain its shape when filled with product to be processed by HPP and to reduce in volume by 0 to at least 30 percent and to withstand a desired number of HPP cycles.

In any of the embodiments described herein, wherein the body has a flexural modulus sufficient to enable the container to retain its shape when filled with product to be processed by HPP and to reduce in volume by 0 to at least 30 percent and to withstand as desired number of HPP cycles.

In any of the embodiments described herein, further comprising at least one opening located on the body other than at the ends of the body.

In any of the embodiments described herein, wherein the body at the location of the at least one opening is recessed to enable a closure positioned at the at least one opening when in closed position to remain within the outer envelope of the body or within the maximum length or width of the body.

In any of the embodiments described herein, comprising a plurality of openings located on the body other than at the ends of the body.

In any of the embodiments described herein, wherein the openings are located on diametrically opposite sides of the body.

In any of the embodiments described herein, further comprising at least two openings located about the container body.

In any of the embodiments described herein, wherein the reusable container has a capacity of from about 20 to at least 250 liters.

In any of the embodiments described herein, wherein the reusable container is sized to be placed into a load basket or any other type of carrier which in turn is used place the reusable container into a pressure vessel.

In any of the embodiments described herein, wherein the container being easily cleaned by introducing cleaning media into, and expelling cleaning media from, the container through the flow valve at the at least one opening.

In any of the embodiments described herein, wherein the closure selected from the group consisting of a cap, a cover, a lid, a plug, a stopper and a valve.

In any of the embodiments described herein, an HPP system comprising:

a high pressure vessel; and

a reusable container according to any previous claim and receivable within the high pressure vessel.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a pictorial view of a modular HPP bulk container of the present disclosure;

FIG. 2 is a side elevational view of a plurality of the modular bulk containers of FIG. 1 arranged in end-to-end relationship; and

FIG. 3 is an enlarged side elevational view of an inlet/outlet valve that may be used with a modular HPP bulk container.

### DETAILED DESCRIPTION

The description set forth below in connection with the appended drawings, where like numerals reference like elements, is intended as a description of various embodiments of the disclosed subject matter and is not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result.

In the following description, numerous specific details are set forth in order to provide a thorough understanding of exemplary embodiments of the present disclosure. It will be apparent to one skilled in the art, however, that many embodiments of the present disclosure may be practiced without some or all of the specific details. In some instances, well known process steps have not been described in detail in order not to unnecessarily obscure various aspects of the present disclosure. Further, it will be appreciated that embodiments of the present disclosure may employ any combination of features described herein.

The present application may include references to “directions,” such as “forward,” “rearward,” “front,” “back,” “ahead,” “behind,” “upward,” “downward,” “above,” “below,” “horizontal,” “vertical,” “top,” “bottom,” “right hand,” “left hand,” “in,” “out,” “extended,” “advanced,” “retracted,” “proximal,” and “distal.” These references and other similar references in the present application are only to assist in helping describe and understand the present disclosure and are not intended to limit the present invention to these directions. The present application may include modifiers such as the words “generally,” “approximately,” “about,” or “substantially.” These terms are meant to serve as modifiers to indicate that the “dimension,” “shape,” “temperature,” “time,” or other physical parameter in question need not be exact, but may vary as long as the function that is required to be performed can be carried out. For example, in the phrase “generally circular in shape,” the shape need not be exactly circular as long as the required function of the structure in question can be carried out.

In the following description and in the accompanying drawings, corresponding systems, assemblies, apparatus and units may be identified by the same part number, but with an alpha suffix. The descriptions of the parts/components of such systems assemblies, apparatus, and units that are the same or similar are not repeated so as to avoid redundancy in the present application.

The present application refers to “processing medium” used in the HPP system for applying high pressure to the product being processed. Such processing medium is also referred to in the application as processing fluid or processing water as well as referred to as pressurized/pressure medium, pressurized/pressure fluid or pressurized water. All of these terms are to be used interchangeably.

In addition, the present application refers to the pressure vessel of an HPP apparatus. Such pressure vessel is also referred to as wire wound vessel or simply vessel. These terms are to be considered as synonymous.

Further, the present application refers to a “product” or “products” that are subjected to or treated by HPP using the containers of the present disclosure. Such product(s) may include all manner of foods, including pumpable foods or beverages, as well as non-food products, such as cosmetics, pharmaceuticals, and organic materials and substances wherein the control of pathogens is desirable.

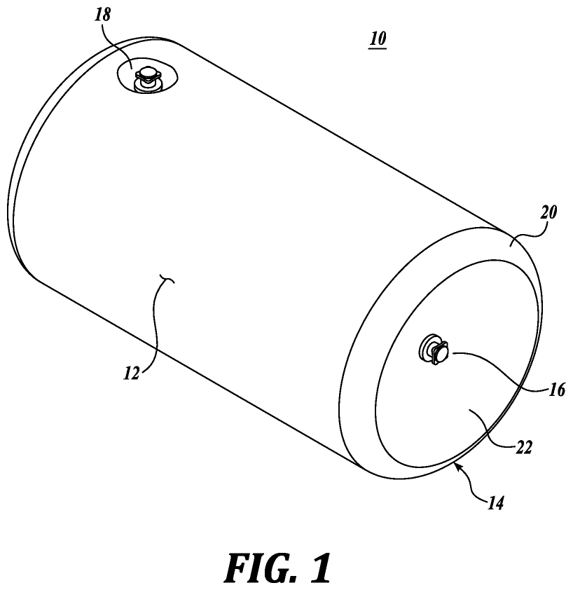

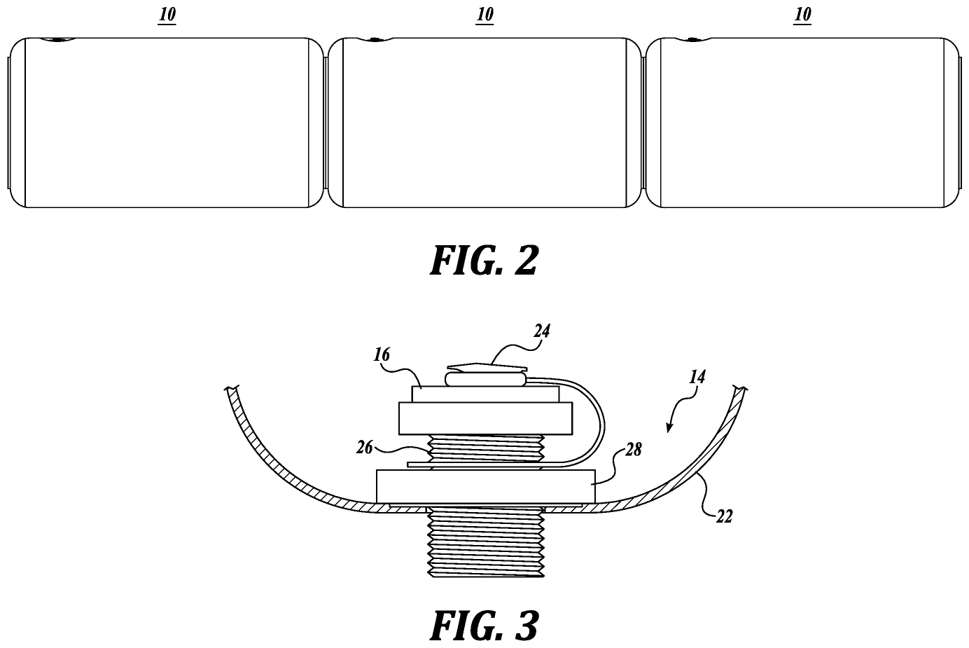

In summary form, as shown in FIG. 1, the bulk HPP container 10 of the present disclosure includes a flexible body portion 12 having closed off ends 14, at least one of which is depicted as being recessed. This enables the containers 10 to be positioned end-to-end in an efficient manner, for example, when placed into an HPP pressure vessel, such as a wire wound vessel, see FIG. 2. One or more openings are provided in the body portion of the container with an appropriate closure or other means for the pumpable produce to enter and exit the container. In this regard, inlet valve 16 or other type of closure may be located at the opening in one or both of the ends of the container. Also, one or more outlet valves 18 or other type of closure are located at an opening on the body 12 of the container for emptying the container, for example, after HPP.

Next, describing the bulk container 10 in more detail, the body 12 is shown as being cylindrical in shape. However, the body can be of other cross-sectional shapes, including pentagonal, hexagonal, octagonal, etc. Also, the body 12 can be of a desired diameter or cross-sectional dimension, as well as of a desired length, so as to provide a desired volume for the bulk container as well as a desired aspect ratio (length v. diameter). Thus, because the bulk containers 10 are of modular design, including the containers being of the same diameter, they can be of different lengths so as to be of various volumes and capacities. In this manner, different beverages or other pumpable products can be processed at the same time using the same pressure level, when the different products may be of different quantities.

As noted above, the bulk container 10 can be of various sizes and volumes. For example, the bulk containers can have a capacity as small as of about 20 to 25 liters, to a capacity of at least 200 to 250 liters or even larger. In this regard, the smallest capacity bulk containers may have a diameter of about 250 to 300 mm or smaller, while the larger containers may have a diameter at least 450 to 475 mm or larger. Of course, the bulk container 10 can be of an even smaller capacity and smaller diameter as well as be of an even larger capacity and a larger diameter. Such smaller bulk containers may be manually filled, placed into and removed from pressure vessel and emptied, whereas the larger containers would require a robot or other lifting and handling system to place the container into the pressure vessel and remove the container from the pressure vessel after processing as well as for filling and emptying the container. As noted above, the bulk containers may be of the same diameter but of different lengths so that different capacity containers may be processed at the same time within the pressure vessel.

It is to be understood that the bulk container 10 can be of size that is appropriate for placing within a load basket of the nature described above. Such load basket is then loadable into the pressure vessel for HPP processing. Once processing has been completed, the bulk container can be conveniently removed from the load basket for emptying and reusing. In this manner, the bulk container need not be of the size that occupies substantially the entire width or the diameter of the interior of the pressure vessel. Rather, this function is assumed by the load basket.

Referring to FIG. 1, the ends 14 of the bulk container 10 have rounded corners 20 that transition into a concave recess 22. Such rounded corners enable the containers to be placed end to end, shown in FIG. 2, without damage to the containers, even if the containers are pushed against each other. Although the smooth rounded corners help prevent damage to the containers, the corners of the containers can be of other shapes and constructions.

The ends 14 of the bulk containers are recessed, with the recess 22 shown in FIG. 1 as being concave in shape. Such recess may constitute a portion of a sphere. An advantage of forming the ends 14 in this manner is that the inlet valve 16 or other type of closure located in the recess remains within the exterior profile of the container, or if the valve does extend beyond the outer envelope, the valve does not extend beyond the overall length or width of the container. Thus, when the bulk containers come into contact with each other or with a wall or other similar surface or abutment, the inlet valve 16 or other type of closure, especially when in closed position, is protected from damage to itself and to adjacent parts. The recess also adds rigidity to the container.

It is to be understood that the bulk container end portions 14 can be of a construction and shape other than as shown in FIG. 1. For example, the end 14 may be substantially planar but with a central recess for receiving the inlet valve 16.

The bulk container 10 can be constructed of various materials, which enable the container to maintain its shape while also being sufficient flexible to adjust to the product inside the container being compressed when the pressure inside the pressure vessel is increased to desired levels during HPP. Such compression and reduction in volume may be from 0 to at least 30%, and perhaps as high as 50%, thereby requiring the volume of the container to be reduced by this same percentage. The material from which the container may be constructed can include, for example, metallic material or polymer material. Such material, as can be appreciated, must be of sufficient flexural strength and sufficient flexural modulus to enable the container to reduce in volume by from 0 to at least 30% while being rigid enough for reuse over a desired number of HPP cycles. Such HPP cycles may be an indefinite number of cycles and so far beyond the number of HPP cycles that occurs in a day, a week, or even in a month. As such, the container can be repeatedly used indefinitely as long as the container is cleaned to meet food cleanliness and/or other applicable standards.

The typical temperature operating range of an HPP operating cycle is from 0° C. to 50° C. However, the operating temperature may be higher when HPP is used in conjunction with heat pasteurization wherein the operating temperature may raise to 65° C. or perhaps 70° C. The material from which the bulk container 10 is constructed is selected to operate within this temperature range, or perhaps at lower or even higher temperatures.

As mentioned above, the bulk container 10 may be composed of a polymer. As a specific non-limiting example, the polymer may be composed of a thermoplastic, such as polyethylene or nylon. As a further non-limiting example, the polymer may be composed of low density polyethylene (LDPE), high density polyethylene (HDPE), or ultra high molecular weight polyethylene (UHMWPE).

As a further non-limiting example, the polymer may have a thickness in the range of from about 4 to 12 mm. The thickness may depend upon several factors, for example, the type of polymer used, the density of the polymer, the diameter of the container, the length of the container, the type of product to be processed, and the pressure level to which the product and container is to be subjected.

The bulk container 10 may be used to process products at high pressures and temperatures than has been the typical operating range for HPP systems. For example, the bulk container may be used operating temperatures of at least 130° C. or higher in situations where both elevated temperatures and pressures are used for sterilization. Such operating pressures may be as high as 8,000 bar or even higher. Many thermal plastics are not designed to operate in these elevated temperatures and pressures. However, “high performance” thermoplastics do exist that are capable to successfully operating at such temperatures and pressures, for example polyetheretherketones, polyamideimides, and polyimides. Also, the thermoplastic may be reinforced with fiberglass or carbon fibers to enhance mechanical and/or thermal properties.

Regardless of the material used to construct the bulk container 10, such material must be compliant with applicable safety standards for food or other products being processed at the operating temperatures being used.

As noted above, the inlet valve 16 or other type of closure may be positioned at an opening in one or both ends 14 of the bulk container 10 in such a manner that the valve, at least when in closed position, is within (does not extend beyond) the outer envelope of the container, or if the valve or other type of closure does extend beyond the outer envelope, or the valve/closure does not extend beyond the overall length or width of the container. This enables the valve/closure to be easily opened and closed, while still protecting the valve from damage, for example, from adjacent containers during HPP.

As a non-limiting example, the valve 16 can be of a plug type valve as shown in FIG. 3. In this regard, the valve 16 includes a closure such as a stopper or plug 24 that frictionally or threadably engages into the valve inlet opening or passageway. The valve stem 26 may be attached or affixed to the container 10 in numerous ways. As one example, the valve stem may be mounted to a mounting base 28 that is in turn affixed to the container 10 by thermal welding, with an adhesive or by other means.

The valve, including the stopper or plug, can be designed and shaped to present a low profile outer contour, thereby reducing the likelihood of damage during handling of the containers as well as during HPP. Moreover, the valve can easily be opened by pulling or unsnapping the valve cap to allow the pumpable material or other liquids or gases to pass through the valve.

It is to be understood that the foregoing is merely one example of a valve or closure that can be used in conjunction with the container 10. Valves of other construction or other types of closures that are suitable for use with the reusable container 10 may also be employed. Further, other types of closures may be used in place of a valve, for example, a cap, a lid, a cover, a plug, a stopper or other device or element that is engageable/insertable within or engageable over or otherwise is capable of closing an opening in the container.

The outlet valve 18 may be of the same or similar construction to the inlet valve 16. Such outlet valve is located at an opening on the container body 12. As shown in FIG. 1, the outlet valve 18 is positioned on the container body at a location of an opening distal from the inlet valve 16. However, the outlet valve may be positioned in other locations, for example, perhaps centrally along the length of the container body 12 or even close to the location of the inlet valve 16. Further, more than one outlet valve may be employed, for example, to facilitate the emptying of the container 10.

In one example of the present disclosure, the container body 12 at the location of the closure, for example, outlet valve 18, is recessed so that when the closure/outlet valve is in closed position or configuration, the closure/valve remains within the outer perimeter or profile (does not extend beyond the outer perimeter or profile) of the container or does not extend beyond the overall length and width (diameter) of the container. As such, the closure/outlet valve, as well as the corresponding opening is protected from damage by undesirable contact with the pressure vessel or other containers or surfaces during filling, during the HPP process, during removal from the pressure vessel, and during other handling of the container, while still being conveniently opened and closed as necessary.

As discussed above, to be understood that the foregoing is merely one example of a valve 18 that can be used in conjunction with the container 10. Valves of other construction that are suitable for use with the reusable container 10 may also be employed. Further, other types of closures may be used in place of a valve, for example, a cap, stopper, or plug engageable/insertable with an opening in the container, or a cap, lid, cover, etc., engageable over, or otherwise capable of closing the opening in the container.

It is anticipated that the bulk container 10 will be filled with a pumpable material, such as a beverage, through an opening, such as the inlet valve 16. During filling, the outlet opening, such as valve 18, is open to expel the air or other gas within the container 10. Of course, all of the inlet and outlet openings or closures, e.g., valves 16 and 18, are closed during HPP. Thereafter, to remove the contents of the container 10, the openings/closures, e.g., valves 16 or 18 are opened. To facilitate the emptying of the container, an opening, perhaps located on the diametrically opposite side of the body 12, may be opened to enable a food approved inert gas to enter the container, such as Nitrogen. Moreover, the gas may be introduced into the container under pressure so as to speed up the emptying process. If a closure, such as valve 18, are placed at openings located on diametrically opposite sides of the body 12, then either valve can be used for emptying the container.

Overall, while valve 16 has been described as an inlet valve, and valve 18 has been described as an outlet valve, the valves 16 and 18 or other type of closures can serve as both outlet and inlet valves/closures. For example, for bulk containers that are filled and emptied while in vertical orientation, the container may be filled and emptied through a closure/valve located at the end of the container. Further, the bulk container may be both filled and emptied using closures/valves positioned on the body of the container, for example, through valve 18 as shown in FIG. 1.

As noted above, the container 10 is designed to be reusable. In this regard, the container 10 is designed to be cleaned as necessary, though perhaps under applicable regulations, such cleaning need not occur after each HPP cycle. Such cleaning can be performed using, for example, hot water, steam, with or without a detergent or other cleaning or disinfectant agent. A cleaning liquid, as well as the subsequent rinsing liquid or fluid, may be introduced into and removed from the container through the valves 16 and 18. Moreover, even if it is not necessary to clean the interior of the container after each usage, the exterior of the container and the valves may be conveniently cleaned due to the shape and construction of the container 10, and the type and positioning of the inlet and outlet closures/valves 16 and 18 described above.

It will be appreciated that the container body 12 is configured to be of sufficient structural integrity to function to contain and hold the product during HPP processing, while also serving as a carrier for the reusable container thereby supporting the reusable container during all stages of high pressure processing, including during filling of the reusable container, during loading of the reusable container into and unloading from a high pressure processing vessel or the load basket, during the high pressure processing of the container contents and during emptying of the reusable container. As a self-contained reusable container and supporting carrier, the container 10 itself can be placed directly into a pressure vessel for HPP processing. No additional or external supporting structure is required to be used with the reusable container 10 of the present disclosure. Further, even with the reusable container performs a carrier function, the container is flexible enough to reduce in volume to match the volume reduction of the product being processed, which reduction can be at least up to 30% of the initial volume of the product. Further, the container 10 is rigid enough to retain its shape (including length and width), during all stages of high pressure processing, as well as being rugged enough for repeated use on an indefinite basis.

It also can be appreciated that by the above construction of the container 10, the contents of the container can be conveniently and safely stored in appropriate facilities both before and after HPP processing, especially at low temperatures, perhaps close to or at 0° C., so as not to permit microbial growth. This is enhanced by the shape and construction of the container 10 for ease of handling, as well as by its material composition.

While illustrative embodiments have been illustrated and described, it will be appreciated that various changes can be made therein without departing from the spirit and scope of the invention. For example, the container 10 is described above as having recessed ends 14. However, rather than both ends of the container 10 being recessed, the container can be constructed so that one end is recessed, and the opposite end is rounded or convex in an outward direction so as to be receivable within the recessed end of an adjacent container. This can facilitate the containers nesting together. In this situation the outwardly rounded or convex end of the container may need to be somewhat recessed in its central portion so as to provide clearance for the valve located in the recessed in portion of an adjacent container.

Further, the container could be constructed so that one or both ends are slanted or diagonal to the length of the container. Moreover, an outlet or inlet can be located at such diagonal so a closure or valve located thereat, though projecting from the exterior surface of the diagonal, is nonetheless, shielded by the protective envelope created the diagonal so the even if the end or side of the container bears against the end of another container or other structure or surface, the closure/valve is not struck. It will be appreciated that in this configuration, the closure/valve is within the overall length and width or diameter of the container.

1. A reusable container for holding pumpable materials during high pressure processing of the material, the container comprising:
(a) a body for receiving the pumpable material and for holding the material during high pressure processing, the body constructed to retain its shape whether empty and filled and having sufficient flexibility to decrease in volume in an amount of at least 30 percent to match the reduction in volume of the held material during high pressure processing;
(b) at least one opening in the body through which the pumpable material is received into the container and/or removed from the container;
(c) a closure at the at least one opening;
(d) the body is configured to be of strength combined with flexibility to function as both reusable container and self-supporting carrier during all stages of high pressure processing and to maintain its diameter, length, and volume properties for a number of high pressure processing cycles that is more than achieved during one day of operation.

2. The reusable container according to claim 1, wherein the closure located at at least one of the openings, when in closed position is within the outer envelope of the container body.

3. The reusable container according to claim 1, wherein the closure located at at least one of the openings does not extend beyond the overall length or width of the container body.

4. The reusable container according to claim 1, wherein the body in cross section is selected from cylindrical, pentagonal, hexagonal, and octagonal.

5. The reusable container according to claim 1, wherein the body defines a length with end portions, and with one or both of the end portions recessed.

6. The reusable container according to claim 5, wherein one or both end(s) of the body are convex in the direction into the body.

7. The reusable container according to claim 5, wherein one or both end(s) of the body are in the shape of a portion of a sphere.

8. The reusable container according to claim 5, wherein an opening is located at at least one end portion of the body.

9. The reusable container according to claim 1, wherein the body is composed of a material selected from the group consisting of a metal and a polymer.

10. The reusable container according to claim 9, wherein the polymer material from which the body is composed is of a material thickness to enable the container to retain its shape when filled with product to be processed by HPP and to reduce in volume by at least 30 percent, or what is needed for the product being processed.

11. The reusable container according to claim 9, wherein the body has a flexural strength sufficient to enable the container to retain its shape when filled with product to be processed by HPP and to reduce in volume by at least 30 percent.

12. The reusable container according to claim 9, wherein the body has a flexural modulus sufficient to enable the container to retain its shape when filled with product to be processed by HPP and to reduce in volume by at least 30 percent.

13. The reusable container according to claim 1, further comprising at least one opening located on the body other than at the ends of the body.

14. The reusable container according to claim 13, wherein the body at the location of the at least one opening is recessed to enable a closure positioned at the at least one opening when in closed position to remain within the outer envelope of the body or within the maximum length or width of the body.

15. The reusable container according to claim 13, comprising a plurality of openings located on the body other than at the ends of the body.

16. The reusable container according to claim 15, wherein the openings are located on diametrically opposite sides of the body.

17. The reusable container according to claim 1, further comprising at least two openings located about the container body.

18. The reusable container according to claim 1, wherein the reusable container has a capacity of from about 20 to at least 250 liters.

19. The reusable container according to claim 1, wherein the container being cleaned by introducing cleaning media into, and expelling cleaning media from, the container through the flow valve at the at least one opening.

20. The reusable container according to claim 1, wherein the closure selected from the group consisting of a cap, a cover, a lid, a plug, a stopper and a valve.

21. A high pressure processing system comprising:
a high pressure vessel; and
a reusable container according to claim 1 and receivable within the high pressure vessel.

22-23. (canceled)
